(19) United States
(12) Patent Application Publication
(10) Pub. No.: US 2024/0041728 A1
(43) Pub. Date: Feb. 8, 2024

(54) ORAL CARE COMPOSITIONS COMPRISING DICARBOXYLIC ACID

(71) Applicant: The Procter & Gamble Company, Cincinnati, OH (US)

(72) Inventors: Samuel James ST. JOHN, Cincinnati, OH (US); Malgorzata Anna KLUKOWSKA, Mason, OH (US); Paul Albert SAGEL, Maineville, OH (US); Tiffany Celeste HARE, West Chester, OH (US); Andrew Frederic GROTH, Mason, OH (US)

(21) Appl. No.: 18/484,713

(22) Filed: Oct. 11, 2023

Related U.S. Application Data
(63) Continuation-in-part of application No. 17/971,728, filed on Oct. 24, 2022.
(60) Provisional application No. 63/275,500, filed on Nov. 4, 2021.

(51) Int. Cl. A61K 8/36; A61K 8/25; A61K 8/21; A61K 8/73; A61Q 11/00

(52) U.S. Cl. CPC A61K 8/36; A61K 8/25; A61K 8/21; A61K 8/731; A61Q 11/00

(57) ABSTRACT

Oral care compositions, such as whitening dentifrice compositions, whitening toothpaste compositions, and/or toothpaste compositions, include dicarboxylic acid, the dicarboxylic acid including malonic acid, methylmalonic acid, dimethylmalonic acid, dihydroxymalonic acid, salts thereof, or combinations thereof, where the composition has a ChemPC ΔL of least about 7.

Mouth rinse compositions include dicarboxylic acid, the dicarboxylic acid including malonic acid, methylmalonic acid, dimethylmalonic acid, dihydroxymalonic acid, salts thereof, or combinations thereof and at least about 75%, by weight of the composition, of water, where the composition has a ChemPC ΔL of least about 7. The composition may be essentially free of, substantially free of, or free of polyphosphate.

### TECHNOLOGICAL FIELD

The present technology relates to oral care compositions comprising dicarboxylic acid with an improved whitening benefit and/or improved iron chelation.

### BACKGROUND

Oral care compositions, such as toothpaste and/or dentifrice compositions, can be applied to the oral cavity to clean and/or maintain the aesthetics and/or health of the teeth, gums, and/or tongue. Additionally, many oral care compositions can be used to remove and/or prevent stains on oral cavity surfaces. Whitening of oral care hard tissue surfaces can occur through chemical or physical means. Physical agents include the combination of a brush and abrasive. Chemical agents include oxidizing agents (e.g., peroxide), anticalculus agents (e.g., polyphosphates), or other agents capable of dislodging surface stains through chemical action (e.g., bicarbonates).

Each agent has their drawbacks. Oxidizing agents are challenging to keep from reacting with other ingredients of the oral care composition during the composition's lifecycle. Additionally, they are not reactive with some surface stains; thereby, not fulfilling their primary purpose. Abrasive agents can cause damage to oral hard tissue surface. Furthermore, they cannot access all areas of the tooth surface where there are stains (e.g., interproximal spaces). Polyphosphate-based anticalculus agents are highly susceptible to hydrolysis breaking down in compositions to ineffective orthophosphate. In the presence of soluble fluoride, the breakdown can be accelerated resulting in insoluble fluoride. Some other chemical agents have characteristic tastes that make them unpleasant to consumers. Bicarbonate-based toothpastes tend to taste like baking soda whose unique experience is not enjoyed by a wide slice of consumers. In total, existing whitening agents can be challenging to formulate with for a variety of reasons specific to each agent.

Thus, there is a need for a whitening agent that can effectively remove and prevent the accumulation of stain, while improving existing formulation challenges.

### SUMMARY

Disclosed herein is a whitening dentifrice composition and/or a whitening toothpaste composition comprising: (a) dicarboxylic acid, the dicarboxylic acid comprising malonic acid, methylmalonic acid, dimethylmalonic acid, dihydroxymalonic acid, salts thereof, or combinations thereof, where the whitening dentifrice composition has a ChemPC ΔL of least about 7 and where the whitening dentifrice composition is essentially free of, substantially free of, or free of polyphosphate.

Also disclosed herein is a toothpaste composition comprising: (a) dicarboxylic acid, the dicarboxylic acid comprising malonic acid, methylmalonic acid, dimethylmalonic acid, dihydroxymalonic acid, salts thereof, or combinations thereof, where the whitening dentifrice composition has a ChemPC ΔL of least about 7 and where the whitening dentifrice composition is essentially free of, substantially free of, or free of polyphosphate.

Also disclosed herein is a mouth rinse composition comprising: (a) dicarboxylic acid, the dicarboxylic acid comprising malonic acid, methylmalonic acid, tartronic acid, malic acid, dimethylmalonic acid, mesoxalic acid, dihydroxymalonic acid, oxalic acid, salts thereof, or combinations thereof; (b) at least about 75%, by weight of the composition, of water, where the composition has a ChemPC ΔL of least about 7 and where the whitening dentifrice composition is essentially free of, substantially free of, or free of polyphosphate.

Also disclosed herein are methods of whitening teeth, removing stain from teeth, and/or preventing the accumulation of stain from teeth comprising applying the disclosed compositions to at least surface of an oral cavity.

### DETAILED DESCRIPTION

Embodiments of the present invention is directed to oral care whitening compositions that have dicarboxylic acid, such as malonic acid, methylmalonic acid, tartronic acid, maleic acid, or combinations thereof. and provide an unexpectedly high stain removal benefit relative to other conventional chemical stain removal agents in a particular pH range. Dental stain, or tooth stain, is caused by the cation-crosslinked proteins and extracellular polysaccharides that then act as reservoirs for colored porphyrins and organic and/or inorganic chromophores. Cross-linking can occur electrostatically via charge-charge, dipole-dipole, and/or dipole-charge interactions. Interrupting these electrostatic forces can facilitate stain removal. The resulting compositions provide efficacious oral hard tissue whitening benefits with fewer drawbacks that are observed with other whitening agents.

Chemical whitening agents loosen the bonds of this colored matrix to affect its removal from the oral hard tissue surfaces. While not wishing to be bound by theory, chemical agents that are effective solubilizing ligands of cationic crosslinking agents in the colored matrix on the oral hard tissue surfaces can be used to remove stain from the surface. Furthermore, the pH and ionic strength of the oral care composition can be used to reduce the strength electrostatic bonds by protonating anionically charged moieties or by reducing the potential of the electrostatic double layer further facilitating the solubilization of cationic moieties by solubilizing ligands (i.e., whitening agents).

The chelate effect postulates that complexes of polydentate ligands with a metal are more stable than the dentate-normalized equivalent of the monodentate-ligand-stabilized metal complex (e.g., 1 mole of a bidentate ligand in comparison to 2 moles of a similarly structured monodentate ligand) because of a reduction in molar entropy of the bidentate chelate with respect to the monodentate complex. The unique properties of dicarboxylate anions allows them, therefore, to be a highly effective stabilizing ligands in a particular pH range. in this way, dicarboxylate anions in a particular pH range are capable of solubilizing and excising metal cations from stained oral enamel and dentin surfaces allowing for facile removal of stained chromogens. While not wishing to be bound by theory, it is believed that the disclosed oral care compositions of the present invention provide an unexpectedly high whitening benefit in comparison to a conventional whitening agent, pyrophosphate.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces. Other examples of oral care compositions include emulsion compositions, such as the emulsions compositions of U.S. Patent Application Publication No. 2018/0133121, jammed emulsions, such as the jammed oil-in-water emulsions of U.S. Pat. No. 11,096,874, and unit-dose compositions, such as the unit-dose compositions of U.S. Patent Application Publication No. 2019/0343732.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients in embodiments of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen’ or “regimen” can be for the use of two or more separate and distinct treatment steps for oral health, e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in embodiments of the present invention. When a range of any type is disclosed or claimed, the intent is to disclose or claim individually each possible number that such a range could reasonably encompass, including end points of the range as well as any sub-ranges and combinations of sub-ranges encompassed therein.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care compositions, as described herein, comprise dicarboxylic acid, tin, and/or fluoride. Additionally, the oral care compositions can comprise other optional ingredients, as described below. The section headers below are provided for convenience only. In some cases, a compound can fall within one or more sections. For example, stannous fluoride can be a tin compound and/or a fluoride compound. Additionally, oxalic acid, or salts thereof, can be a dicarboxylic acid, a polydentate ligand, and/or a whitening agent.

### Dicarboxylic Acid

The oral care composition comprises dicarboxylic acid. The dicarboxylic acid comprises a compound with two carboxylic acid functional groups. The dicarboxylic acid can comprise a compound or salt thereof defined by Formula I-A, Formula I-B, and/or Formula I-C.

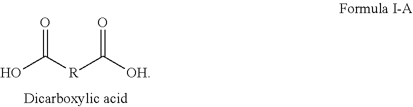

R can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

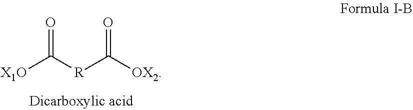

R can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

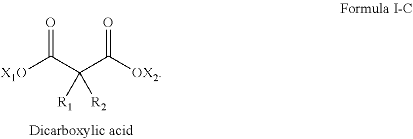

R₁ can be null, alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

The dicarboxylic acid can be added to a formulation as a neutral acid (as shown in Formula I-A) or as a dicarboxylate monosalt (where one of the carboxylic acid functional groups is a salt and the other is neutral), a dicarboxylate disalt (where both of the carboxylic acid functional groups are salts), or combinations thereof. Additionally, as is well known to a person of ordinary skill in the art, whether or not that one or both of the carboxylic acid functional groups of the dicarboxylic acid are neutral or charged in solution, can be influenced by the pH of the solution. For example, a neutral dicarboxylic acid can be added to an aqueous solution and one or two protons from the two carboxylic acid functional groups can be removed if the pH is lower than the pKa of the carboxylic acid functional group, as shown below in Formula I-D.

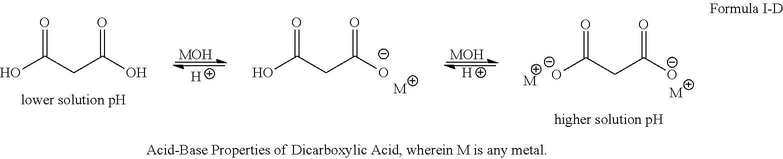

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, dihydroxymalonic acid, fumaric acid, terephthalic acid, glutaric acid, salts thereof, or combinations thereof. The dicarboxylic acid can comprise suitable salts of dicarboxylic acid, such as, for example, when the dicarboxylic acid includes a salt of oxalic acid: monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

Suitable dicarboxylic acid compounds include malonic acid, methylmalonic acid, tartronic acid, malic acid, dimethylmalonic acid, mesoxalic acid, dihydroxymalonic acid, oxalic acid, salts thereof, or combinations thereof. These dicarboxylic acid compounds are particularly suitable as these compounds have been shown to have an unexpectedly high whitening benefit. While not wishing to be bound by theory, it is believed that particular dicarboxylic acid compounds have an unexpectedly high affinity to certain cationic crosslinking agents typically found in the colored matrix on the oral hard tissue surfaces, thereby resulting in the removal of stain from the surface.

Suitable dicarboxylic acid compounds include dicarboxylic acids described by Formula I-A, wherein R is null, comprises a methylene or ethylene with one or two substitutions, and/or an acetyl group.

Without being bound by theory, it is hypothesized that the whitening efficacy of the dicarboxylics acids and their corresponding anions is driven by the ability of the dicarboxylic acid to reach and remove cationic bridges between chromophores and the tooth surface as well as chromophores and the pellicle proteins. It is further believed that iron plays a key role in linking chromophores to surfaces and surface bound proteins. Dicarboxylic acid chelation can be strong enough to remove iron bridges enabling the stain to be rinsed away, detached from the tooth surface, or be more easily removed by friction or other means of mechanical cleaning such as toothbrushing.

Iron can be of interest for gum health and overall body health through a multitude of cascading mechanisms. It is well known to a person of ordinary skill in the art that iron is an essential element for most living organisms, such as gram negative anaerobic oral bacteria. For example, Porphyromonas gingivalis, Prevotella intermedia, and Tannerella forsythia are gram negative anaerobic bacteria associated with the cause and the progression of gum disease. These bacteria along with other oral bacteria have mechanisms to regulate iron to enable their survival and growth in the oral cavity, such as within biofilms and below the gum line in the gingival pockets, which are typically considered anaerobic locations. Additionally, iron upregulates virulence gene expressions that in turn leads to host tissue barrier disruption and bacterial invasion at both tissue and cellular levels. Generally, the human body does not maintain sufficient levels of bioavailable iron to support the viability of pathogenic bacteria. Hence these bacteria utilize biological chelators such as siderophores, iron binding proteins, porphyrins (heme and hemoproteins) to recruit, retain and transfer iron into the bacteria. Thus, the overall virulence and infection potential of gram-negative oral anaerobes can depend in part on the availability of iron from these chelation systems. While not wishing to being bound by theory, it is believed that overcoming the bacterial iron recruiting and retention mechanisms can enable a shift of the available iron out of and away from these pathogenic bacteria, thereby limiting metabolic activity and down regulating gene expressions associated with the progression of gum disease leading to an improvement in gum health and overall health.

As further described herein. dicarboxylic acids can be chelators with a relatively small molecular size that can have the ability to chelate iron away from gram negative organisms that can lead to overall improved gum health. Suitable examples of dicarboxylic acids that can improve oral health through iron controls are malonic acid, methyl malonic acid, dimethyl malonic acid, D-tartaric acid, L-tartaric acid, DL tartaric acid, oxalic acid, tartonic acid, mesoxalic acid, dihydroxymalonic acid, maleic acid, malic acid, succinic acid, fumaric acid. Other examples with longer hydrophobic chains include glutaric acid, adipic acid, pimetic acid, suberic acid, aszelaic acid, sebacic acid, undecanedioic acid, dodecanoic acid, brassylic acid, tetradecanedioic acid, pentadecanedioic acid, thaspic acid, octadecanedioic acid. While not wishing to being bound by theory, it is believed that dicarboxylic acids with increased hydrophobicity can demonstrate greater penetration of the hydrophobic portions of the bacterial wall structures, which can further reduce the iron availability to the anaerobes.

The oral care composition can comprise from about 0.01% to about 10%, from about 0.1% to about 15%, from about 1% to about 5%, or from about 0.0001 to about 25%, by weight of the oral care composition, of dicarboxylic acid. The wt % of the dicarboxylic acid in a composition can be relative to the dicarboxylic acid molecule itself, excluding any metallic counter ions that may be present.

The oral care composition can also comprise from about 2% to about 10%, greater than 2% to about 10%, from about 2.5% to about 5%, or about 3% to 10%, by weight of the oral care composition, of dicarboxylic acid. The wt % of the dicarboxylic acid in a composition can be relative to the dicarboxylic acid molecule itself, excluding any metallic counter ions that may be present.

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, K, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Tin

The oral care composition according to embodiments of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, or from about 0.3% to about 0.6%, by weight of the dentifrice composition. Alternatively, the oral care composition can be essentially free of, substantially free of, or free of zinc.

### Potassium

The oral care composition can comprise potassium, which can be provided by a potassium ion source. The potassium ion source can comprise one or more potassium containing compounds, such as potassium nitrate, potassium fluoride, potassium chloride, or combinations thereof.

The oral care composition can comprise from about 0.01% to about 10%, from about 0.2% to about 1%, from about 0.4% to about 1%, or from about 0.3% to about 0.6%, by weight of the oral care composition, of potassium and/or potassium ion source. Alternatively, the oral care composition can be essentially free of, substantially free of, or free of potassium.

### Quaternary Ammonium Compound

The oral care composition can include quaternary ammonium compound. The quaternary ammonium compounds in the compositions according to embodiments of the present invention can include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Cetylpyridinium chloride, cetyl pyridinium fluoride, tetradecylpyridinium chloride, N-tetradecyl-4-ethyl pyridinium chloride, domiphen bromide, benzalkonium chloride, benzethonium chloride, methyl benzethonium chloride, dodecyl trimethyl ammonium bromide, dodecyl dimethyl (2-phenoxyethyl) ammonium bromide, benzyl dimethoxystearyl ammonium chloride, quaternized 5-amino-1,3-bis(2-ethyl-hexyl)-5-methyl hexa hydropyrimidine, lauryl trimethylammonium chloride, cocoalkyl trimethylammonium chloride, cetyl trimethylammonium bromide, di-isobutylphenoxyethyl-dimethylbenzylammonium chloride, dodecyl trimethyl ammonium bromide, are exemplary of typical quaternary ammonium antimicrobial agents. Other compounds are bis[4-(R-amino)-1-pyridinium] alkanes as disclosed in U.S. Pat. No. 4,206,215 to Bailey. The pyridinium compounds are the preferred quaternary ammonium compounds, particularly preferred being cetylpyridinium, or tetradecylpyridinium halide salts (i.e., chloride, bromide, fluoride and iodide). Particularly preferred are cetylpyridinium chloride and fluoride salts.

The oral care composition can comprise at least about 0.025%, at least about 0.035%, at least about 0.045% to about 1.0%, from about 0.025% to about 1%, or from about 0.01% to about 10%, by weight of the composition, of the quaternary ammonium compound.

pH

The pH of the oral care compositions as described herein can be from about 4 to about 7, from about 4 to about 6, from about 4.5 to about 6.5, from about 4.5 to about 5.5, from about 4 to less than 5.5, from about 4.5 to less than 5.5, greater than 4 to less than 5, greater than 4 to about 4.9, from about 4.9, from about 4 to about 5.4, from about 4 to about 5.3, from about 4 to about 5.2, from about 4 to about 5.1, from about 4 to about 5, from about 4 to about 4.9, from about 4 to about 4.8, from about 4 to about 4.7, or from about 4.8 to about 5.3. The pH of a mouthrinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

The pH of the oral care compositions as described herein have a preferred pH of below about 7 or below about 6 due to the pKa of the dicarboxylic acid. While not wishing to be bound by theory, it is believed that the dicarboxylic acid displays unique behavior when the pH is below about 7 or below about 6, but surfaces in the oral cavity can also be sensitive to a low pH. Additionally, at pH values above about pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity.

Additionally, at pH values less than 4, the potential for demineralization is greatly increased. Consequently, the oral care compositions comprising dicarboxylic acid, as described herein, can preferably have a pH from about 4 to about 7, from about 4 to about 6, from about 4.5 to about 6.5, from about 4 to about 5, from about 4 to less than 5, from about 4 to about 4.9, or from about 4.5 to less than 5.5 to minimize metal hydroxide/metal oxide formation and any increased demineralization in the oral cavity).

The pH of the oral care composition, as described herein, can be measured either immediately upon mixing, or upon aging the composition by placing the oral care composition at ambient or accelerated temperature and humidity conditions, such as including measuring the pH at a temperature of 25° C., 30° C., and/or 40° C. with a 30%, 60% and/or 75% relative humidity for about 28 days or longer prior to measuring the pH.

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of from about 0.1% to about 30%, from about 1% to about 10%, or from about 1.5% to about 3%, by weight of the present composition.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in embodiments of the present invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about I to about 20%, or about 10% or less, by weight of the oral care composition, of the polyphosphate source. Alternatively, the oral care composition can be essentially free of, substantially free of, or free of polyphosphate.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about to about 10%, or from about 0.3% to about 2.5%, by weight of the oral care composition.

### Monodentate Ligand

The oral care composition can comprise monodentate ligand having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can comprise a single carboxylic acid functional group. Suitable monodentate ligands comprising carboxylic acid can include compounds with the formula R—COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The oral care composition can include from about 0.01% to about 10%, from about 0.1% to about 15%, from about 1% to about 5%, or from about 0.0001 to about 25%, by weight of the composition, of the monodentate ligand.

### Polydentate Ligand

The oral care composition can comprise polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e. with two functional groups), tridentate (i.e. with three functional groups), tetradentate (i.e. with four functional groups), etc.

Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

The polydentate ligand can comprise two or more carboxylic acid functional groups. Suitable polydentate ligands comprising carboxylic acid can include compounds with the formula HOOC—R—COOH, wherein R is any organic structure. Suitable polydentate ligands comprising two or more carboxylic acid can also include dicarboxylic acid, tricarboxylic acid, tetracarboxylic acid, etc.

Other suitable polydentate ligands include compounds comprising at least two phosphate functional groups. Thus, the polydentate ligand can comprise polyphosphate, as described herein.

Other suitable polydentate ligands include hops beta acids, such as lupulone, colupulone, adlupulone, and/or combinations thereof. The hops beta acid can be synthetically derived and/or extracted from a natural source.

The polydentate ligand can also include phosphate as the functional group to interact with the tin. Suitable phosphate compounds include phosphate salts, organophosphates, or combinations thereof. Suitable phosphate salts include salts of orthophosphate, hydrogen phosphate, dihydrogen phosphate, alkylated phosphates, and combinations thereof. The polydentate ligand can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, maleic acid, malic acid, tartaric acid, phthalic acid, citric acid, phytic acid, pyrophosphate, tripolyphosphate, tetrapolyphosphate, hexametaphosphate, salts thereof, and/or combinations thereof.

The oral care composition can include from about 0.01% to about 10%, from about 0.1% to about 15%, from about 1% to about 5%, or from about 0.0001 to about 25%, by weight of the composition, of the polydentate ligand.

### Ratio of Tin to Monodentate Ligand to Polydentate Ligand

The oral care composition, as described herein, can comprise a ratio of tin to monodentate ligand to polydentate ligand that provides an unexpectedly high amount of soluble tin and/or a superior fluoride uptake. Suitable ratios of tin to monodentate ligand to polydentate ligand can be from about 1:0.5:0.5 to about 1:5:5, from about 1:0.5:0.75 to about 1:5:5, from about 1:1:1 to about 1:5:5, from about 1:1:0.5 to about 1:2.5:2.5, from about 1:1:1 to about 1:2:2, from about 1:0.5:0.5 to about 1:3:1, or from about 1:0.5:0.5 to about 1:1:3.

Desired herein are oral care compositions with a soluble Sn of at least about 1000 ppm, 2000 ppm, 4000 ppm, at least about 4500 ppm, at least about 5000 ppm, at least about 6000 ppm, and/or at least about 8000 ppm. Also desired herein are oral care compositions with a fluoride uptake of at least about 6.5 μg/cm², at least about 7.0 μg/cm², at least about 8.0 μg/cm², or at least about 9.0 μg/cm² after a time period of at least about 9 days, 30 days, 65 days, 75 days, 100 days, 200 days, 365 days and/or 400 days.

In total, while not wishing to be bound by theory it is believed that the soluble Sn amount is correlated to bioavailable Sn as it is freely available to provide an oral health benefit. Fully bound Sn (i.e. Sn that is overchelated) or precipitated Sn (i.e. insoluble tin salts, such as Sn(OH)₂ and/or Sn-based stains can form when Sn is underchelated) would not be included in the measurement for soluble Sn. Additionally, while not wishing to be bound by theory, it is believed that a carefully balanced ratio of Sn to monodentate and polydentate ligands can provide a high amount of bioavailable fluoride and Sn ions without some of the negatives to the use of cationic antimicrobial agents, such as surface staining. Thus, additional screening experiments were done to quantify and qualify the ranges and identities of monodentate and polydentate ligands.

### Thickening Agent

The oral care composition can comprise one or more thickening agents. Thickening agents can be useful in the oral care compositions to provide a gelatinous structure that stabilizes the toothpaste against phase separation. Suitable thickening agents include polysaccharides, polymers, and/or silica thickeners. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas. The oral care composition can comprise from 0.01% to about 15%, from 0.1% to about 10%, from about 0.2% to about 5%, or from about 0.5% to about 2% of one or more thickening agents.

### Abrasive

The oral care composition according to embodiments of the present invention can comprise an abrasive. Abrasives can be added to oral care formulations to help remove surface stains from teeth. Preferably, the abrasive is a calcium abrasive or a silica abrasive.

The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

The oral care composition can also comprise another abrasive, such as bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. The oral care composition can comprise from about 5% to about 70%, from about 10% to about 60%, from about 10% to about 50%, from about 20% to about 50%, from about 25% to about 40%, or from about 1% to about 50% of another abrasive.

The oral care composition can include a low level of or no abrasive as the dicarboxylic acid can provide a high enough whitening benefit that an abrasive is not necessary. While mouth rinse compositions typically do not include abrasive, dentifrice compositions typically do include abrasive. However, the dentifrice compositions and/or toothpaste compositions according to embodiments of the present invention can include a low level of or no abrasive. As such, the dentifrice composition can comprise less than about 5%, from about 0.5% to about 2%, or less than about 2%, by weight of the oral care composition, of abrasive. The dentifrice composition can also be essentially free of, substantially free of, or free of abrasive.

### Amino Acid

The oral care composition can comprise amino acid. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula II, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

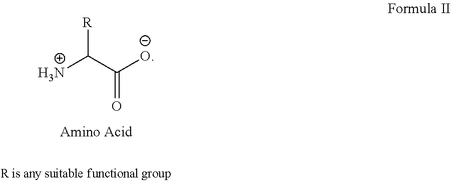

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in embodiments of the present invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in embodiments of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, pthalamidoperoxycaproic acid, or mixtures thereof.

The whitening dentifrice composition can be essentially free of, substantially free of, or free of peroxides, metal chlorites, perborates, percarbonates, peroxyacids, persulfates, and combinations thereof. For example, the whitening dentifrice composition can be essentially free of, substantially free of, or free of peroxide, chlorohexidine, and bicarbonate.

The whitening agent may consist essentially of dicarboxylic acids or salts thereof, or the whitening agent may consist of dicarboxylic acids or salts thereof. The whitening agent may consist essentially of malonic acid or a salt thereof, or the whitening agent may consist of malonic acid or a salt thereof. The oral care composition may comprise 90% or greater, 95% or greater, or 99% or greater, by weight of the whitening agent, of one or more dicarboxylic acids or salts thereof. The oral care composition may comprise 90% or greater, 95% or greater, or 99% or greater, by weight of the whitening agent, of malonic acid or the salt thereof.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition according to embodiments of the present invention can be a dentifrice composition that is anhydrous, a low water formulation, or a high water formulation. In total, the oral care composition can comprise from 0% to about 99%, about 20% or greater, about 30% or greater, about 50% or greater, up to about 45%, or up to about 75%, by weight of the composition, of water. Preferably, the water is USP water.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions for the delivery of the dicarboxylic acid include emulsion compositions, such as the emulsions compositions of U.S. Patent Application Publication No. 2018/0133121, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, such as the jammed oil-in-water emulsions of U.S. Pat. No. 11,096,874, which is herein incorporated by reference in its entirety, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Methods

The oral care composition comprising dicarboxylic acid, as described herein, can lead to oral health benefits, such as the whitening of teeth, removing stain from teeth, and/or preventing the accumulation of stain from teeth when applied to the oral cavity. For example, a user can dispense at least a one-inch strip of a suitable oral care composition, as described herein, onto an oral care implement, such as a toothbrush, applicator, and/or tray, and applied to the oral cavity and/or teeth.

The user can be instructed to brush teeth thoroughly for at least 30 seconds, at least one minute, at least 90 seconds, or at least two minutes at least once, at least twice, or at least three times per day. The user can also be instructed to expectorate the oral care composition after the completion of the brush procedure.

The user can also be instructed to rinse with a mouthwash composition comprising dicarboxylic acid and/or mouth rinse composition comprising dicarboxylic acid after the completion of the brush procedure or instead of the brush procedure. The user can be instructed to swish the oral care composition thoroughly for at least 30 seconds, at least one minute, at least 90 seconds, or at least two minutes at least once, at least twice, or at least three times per day. The user can also be instructed to expectorate the oral care composition after the completion of the procedure.

The oral care compositions according to embodiments of the present invention can be used to provide a whitening benefit, such as the whitening of teeth, removing stain from teeth, and/or preventing the accumulation of stain from teeth. The oral care compositions useful for the methods include dicarboxylic acid, as described above, and optionally metal, quaternary ammonium compound, and/or fluoride.

The oral care composition can include primary packaging, such as a tube, bottle, and/or tub. The primary package can be placed within secondary package, such as a carton, shrink wrap, or the like. Instructions for use of the oral care composition can be printed on the primary package and/or the secondary package. The scope of the method is intended to include instructions provided by a manufacturer, distributor, and/or producer of the oral care composition.

If the oral care composition is a toothpaste, the user can be instructed to dispense the toothpaste from the toothpaste tube.

The user can be instructed to apply a portion of the toothpaste onto a toothbrush. The portion of the toothpaste can be of any suitable shape, such as strip, a pea-sized amount, or various other shapes that would fit onto any mechanical and/or manual brush head. The user can be instructed to apply a strip of the toothpaste that is at least about 1 inch, at least about 0.5 inch, at least 1 inch, and/or at least 0.5 inch long to the bristles of a toothbrush, such as soft-bristled toothbrush.

The user can be instructed to apply pea-sized or grain of rice-sized portion of the toothpaste to the bristles of a toothbrush, such as in the case of use by children of less than 6 years old and/or less than 2 years old.

The user can be instructed to brush their teeth for at least about 30 seconds, at least about 1 minute, at least about 90 seconds, at least about 2 minutes, at least 30 seconds, at least 1 minute, at least 90 seconds, and/or at least 2 minutes.

The user can be instructed to brush their teeth thoroughly and/or as directed by a physician and/or dentist.

The user can be instructed to brush their teeth after each meal. The user can be instructed to brush their teeth at least once per day, at least twice per day, and/or at least three times per day. The user can be instructed to brush their teeth no more than three times a day, such as to prevent Sn staining. The user can be instructed to brush their teeth in the morning and/or in the evening prior to sleeping.

The user can be instructed to not swallow the toothpaste composition due to the inclusion of ingredients that are not suitable for ingestion, such as fluoride. The user may be instructed to expectorate (or spit out) the toothpaste composition after the cessation of the brushing cycle.

If the oral care composition is a mouth rinse, the user can be instructed to dispense the mouth rinse from a bottle containing the mouth rinse.

The user can be instructed to use the mouth rinse at least once a day, at least twice a day, and/or at least three times a day.

The user can be instructed to use the mouth rinse composition after the use of toothpaste and/or floss.

The user can be instructed to swish a portion of rinse in the oral cavity, such as between the teeth, for a period of time. The user can be instructed to vigorously swish a portion of the rinse.

The user can be instructed to use be from about 5 mL to about 50 mL, from about 10 mL to about 40 mL, 10 mL, 20 mL, 25 mL, 30 mL, 40 mL, 2 teaspoonfuls, and/or 4 teaspoonfuls of mouth rinse.

The user can be instructed to swish the mouth rinse for at least about 30 seconds, at least about 1 minute, at least about 90 seconds, at least about 2 minutes, at least 30 seconds, at least 1 minute, at least 90 seconds, and/or at least 2 minutes.

The user can be instructed to not swallow the mouth rinse composition due to the inclusion of ingredients that are not suitable for ingestion, such as fluoride. The user may be instructed to expectorate (or spit out) the mouth rinse composition after the cessation of the rinse cycle.

The usage instructions for the oral care composition, such as for a toothpaste composition and/or a mouth rinse composition, can vary based on age. For example, adults and children that are at least 6 or at least 2 can have one usage instruction while children under 6 or under 2 can have a second usage instruction.

Suitable medicaments include oral care compositions, toothpaste compositions, mouth rinse compositions, floss coatings, chewing gums, and/or other suitable compositions to be applied in the oral cavity.

### Oral Care Regimen

The dicarboxylic acid can be delivered in the same composition as the tin and/or fluoride or the dicarboxylic acid can be delivered in a separate composition. For example, a first composition can comprise tin and/or fluoride and a second composition can comprise dicarboxylic acid. The first and second composition can be delivered simultaneously, such as in a dual-phase composition or sequentially from discrete compositions.

An oral care kit can include the first composition comprising tin and/or fluoride and the second composition comprising dicarboxylic acid. The oral care kit can also include instructions directing a user to apply the first composition to an oral cavity of the user followed by applying the second composition to the oral cavity of the user. The first composition can be expectorated prior to the application of the second composition or the second composition can be applied prior to the expectoration of the first composition from the oral cavity.

The entire oral care regimen can have a duration of from one minute to about three minutes with each application step having a duration of from about 30 seconds to about 2 minutes or about 1 minute.

The components can be delivered to the oral cavity simultaneously or sequentially. The simplest case is simultaneous, continuous delivery of equal amounts of the two components or a constant ratio of the components during a single oral care session. The two components may be provided separately, such as in a dual-phase composition in two separate compositions, and then delivered simultaneously to the oral cavity. Brushing duration is sufficiently short so that the components will not be inactivated. Another use for simultaneous, continuous delivery is systems that include two components that react relatively slowly, and that will remain in the oral cavity after brushing to be absorbed by the teeth and or gums.

In the case of sequential delivery, both components may be delivered during a single oral care session, e.g., a single brushing session or other single treatment session (single use, start to finish, by a particular user, typically about 0.1 to 5 minutes), or alternatively the components may be delivered individually over multiple oral care sessions. Many combinations are possible, for example delivery of both components during a first oral care session and delivery of only one of the components during a second oral care session.

Sequential delivery during a single oral care session may take various forms. In one case, two components are delivered in alternation, as either a few relatively long duration cycles during brushing (A B A B), or many rapid-fire alternations (A B A B A B A B A B . . . A B).

In another case, two or more components are delivered one after the other during a single oral care session, with no subsequent alternating delivery in that oral care session (A followed by B). For example, a first composition comprising fluoride and/or tin can be delivered initially, to initiate brushing and provide cleansing, followed by a second composition comprising dicarboxylic acid.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Example 1

The indicated quantity of dicarboxylic acid or salt of dicarboxylic acid was combined with 20.00 g of ultra-pure (18.2 MΩ) water and neutralized dropwise with 1N HC1 or 1N NaOH to obtain a pH of about 5. The solution of dicarboxylate in water was then combined with 20.00 g of toothpaste with vigorous, non-aerating agitation to obtain a slurry of toothpaste, water, and dicarboxylic acid or salt of dicarboxylic acid. The pH was checked a final time and adjust to about 5 dropwise if needed. The compositions were compared to a conventional whitening toothpaste with pyrophosphate whitening agent, Crest Tartar Protection.

TABLE 1A
Oral Care Compositions

| Ingredient (g) | Ex. 1 | Ex. 2 | Ex. 3 | Ex. 4 | Ex. 5 | Ex. 6 | Ex. 7 |
|---|---|---|---|---|---|---|---|
| Malonic Acid | 0.71 | 0 | 0 | 0 | 0 | 0 | 0 |
| Methylmalonic Acid | 0 | 0.80 | 0 | 0 | 0 | 0 | 0 |
| Tartronic Acid | 0 | 0 | 0.82 | 0 | 0 | 0 | 0 |
| Malic Acid | 0 | 0 | 0 | 0.91 | 0 | 0 | 0 |
| Dimethylmalonic Acid | 0 | 0 | 0 | 0 | 0.90 | 0 | 0 |
| Sodium Mesoxalate | 0 | 0 | 0 | 0 | 0 | 1.23 | 0 |
| Monohydrate¹ | | | | | | | |
| Potassium Oxalate | 0 | 0 | 0 | 0 | 0 | 0 | 1.26 |
| Monohydrate | | | | | | | |
| Toothpaste Composition 1 | 20.00 | 20.00 | 20.00 | 20.00 | 20.00 | 20.00 | 20.00 |
| Ultra-Pure Water | 20.00 | 20.00 | 20.00 | 20.00 | 20.00 | 20.00 | 20.00 |

¹Sodium Mesolxalate Monohydrate may tautomerize to include at least a portion of dihydroxymalonic acid upon contact with water.

TABLE 1B
Oral Care Compositions

| Ingredient (g) | Ex. 8 | Ex. 9 | Ex. 10 | Ex. 11 | Ex. 12 | Ex. 13 |
|---|---|---|---|---|---|---|
| DL Tartaric Acid | 1.02 | 0 | 0 | 0 | 0 | 0 |
| L+ Tartaric Acid | 0 | 1.02 | 0 | 0 | 0 | 0 |
| Phthalic Acid | 0 | 0 | 1.13 | 0 | 0 | 0 |
| D- Tartaric Acid | 0 | 0 | 0 | 1.02 | 0 | 0 |
| Succinic Acid | 0 | 0 | 0 | 0 | 0.80 | 0 |
| Fumaric Acid | 0 | 0 | 0 | 0 | 0 | 0.79 |
| Toothpaste | 20.00 | 20.00 | 20.00 | 20.00 | 20.00 | 20.00 |
| Composition 1 | | | | | | |
| Ultra-Pure Water | 20.00 | 20.00 | 20.00 | 20.00 | 20.00 | 20.00 |

TABLE 1C
Oral Care Compositions

| | Toothpaste | Toothpaste |
|---|---|---|
| | Composition 1 | Composition 2 |
| Name | (wt %) | (wt %) |
| Sorbitol (70%) | — | 63.408 |
| Glycerin 99.7% veg base | 40 | |
| Treated Water | 36.53 | 8.024725 |
| Sodium Fluoride | — | 0.243 |
| Sodium Hydroxide (50% active, aqueous solution) | — | 2.3 |
| Saccharin | — | 0.414 |
| Stevia | 0.5 | — |
| Xanthan Gum | 0.7 | 0.7 |
| Carboxymethyl cellulose | — | 0.2 |
| Carbomer | — | 0.3 |
| Carrageenan | 0.7 | — |
| Sodium Acid Pyrophopshate | — | 4.17 |
| Dye | — | 0.000275 |
| Titanium Dioxide | 0.5 | 0.25 |
| Silica Z165 | 2 | — |
| Silica Z109 | 7 | — |
| Silica Z119 | 10 | 15 |
| Sodium Lauryl Sulfate (29% active, aqueous solution) | — | 4 |
| Tego Betaine | 1.35 | — |
| Flavor | 0.72 | 0.99 |

### Chemistry-Only Pellicle Cleaning (ChemPC)

The method of the conventional pellicle cleaning ratio (PCR) is a well-accepted industry method to investigate the whitening properties of abrasive-containing compositions as a means to estimate their clinical stain removal potential. The method was originally published by Stookey et al. (1982) and was later refined by Schemehorn et al. (2011) to make a darker, more tenacious stain. The method of Schemehorn et al. was used here to produce the stained chips in this chemistry-only pellicle cleaning (ChemPC) study to evaluate the ability of the dicarboxylate-containing formulations to remove a dental stain mimic. Their stain removal efficacy was determined by their ability to remove the dental stain mimic measured as a change in L value from each specimen's baseline color value in the CIE L*′a*b*color scale on an average of eight chips per treatment group.

In this experiment, approximately 40 g of toothpaste slurry from TABLE 1A and TABLE 1B was added to a 50 mL Falcon tube (Falcon 50 mL, 115 mm Long Conical Centrifuge Tubes, Fisher Scientific, Thermo Fisher Scientific, Waltham, MA, USA) inside which four pre-imaged, stained PCR enamel chips (Therametric Technologies, Inc., Noblesville, IN) with dental stain mimic had been placed. Two tubes comprising 8 chips and 80 g of total slurry were needed for each treatment group. The tubes were then loaded into a rotator/tumbler (Cole-Parmer Instrument Company; Serial: 558501, Chicago, IL) for overnight tumbling. The rotator was turned on, set to a speed setting of 4.5, and allowed to tumble undisturbed for 16 hours. After 16 hours of tumbling, the chips were removed, rinsed with ultra-pure water, and post imaged. The ΔL value was determined by subtracting the pre-treatment image L value from the post-treatment image L value. The ΔL values obtained herein are reported in TABLE 2.

Tooth color is measured using a digital camera having a lens equipped with a polarizer filter (Camera model no. CANON EOS 70D from Canon Inc., Melville, NY with NIKON 55 mm micro-NIKKOR lens with adapter). The light system is provided by Dedo lights (model number DLH2) equipped with 150 watt, 24V bulbs model number (Xenophot model number HL X64640), positioned about 30 cm apart (measured from the center of the external circular surface of one of the glass lens through which the light exits to the other) and aimed at a 45 degree angle, such that the light paths intersect at the vertical plane of the sample holder about 36 cm in front of the focal plane of the camera. Each light has a polarizing filter (Lee 201 filter), and a cutoff filter (Rosco 7 mil Thermashield filter from Rosco, Stamford, CT, USA).

At the intersection of the light paths, a fixed sample holder is mounted for reproducible repositioning in the light field. The camera is placed between the two lights such that its focal plane is about 36 cm from the vertical plane of the chin rest. Prior to beginning the measurement of tooth color, color standards are imaged to establish calibration set-points. A Munsell N8 grey standard is imaged first. The white balance of the camera is adjusted, such that the RGB values of grey are 200. Color standards are imaged to get standard RGB values of the color chips. The color standards and grey standard are listed below (from Munsell Color, Division of X-rite, Grand Rapids, MI, USA). Each color standard is labeled with the Munsell nomenclature. To create a grid of color standards they can be arranged in the following manner. This enables multiple color standards to be contained in a single image captured of the grid of color standards.

Color standard grid 1

| | | | | | |
|---|---|---|---|---|---|
| 7.5R 6 8 | 2.5R 6 10 | 10YR 6.5 3 | POLARIZATION | 5R 7 8 | N 3.5 0 |
| | | | CHECK | | |
| 7.5RP 6 6 | 10R 5 8 | 5YR 7 3 | 2.5Y 8.5 2 | 2.2YR 6.47 4.1 | 7.5YR 7 4 |
| 5YR 8 2 | N 8 0 | 10R 7 4 | N 8 0 | 5YR 7.5 2.5 | 2.5Y 8 4 |
| 5YR 7 3.5 | 5YR 7 2.5 | 5YR 5 2 | 5YR 7.5 2 | N 6.5 0 | N 9.5 0 |

Color standard grid 2

| | | | | | |
|---|---|---|---|---|---|
| 5YR 7.5 3.5 | 2.5Y 6 4 | 10YR 7.5 3.5 | 2.5R 7 8 | 7.5R 7 8 | 10YR 7.5 2 |
| 10YR 7.5 2.5 | N 5 0 | 2.5R 6 8 | 10YR 7 2 | 5R 7 4 | 10YR 7 2.5 |
| N 6.5 0 | 7.5RP 6 8 | 7.5R 8 4 | 5Y 8 1 | 7.5YR 8 2 | 2.2YR 6.47 4.1 |
| N 5 0 | 2.5Y 8 4 | 10YR 7 3 | N 9.5 0 | 10RP 7 4 | 2.5Y 7 2 |

Color standard grid 3

| | | | | | |
|---|---|---|---|---|---|
| 5R 6 10 | N 8.5 0 | 10YR 6.5 3.5 | 10RP 6 10 | N 8 0 | 7.5YR 7 3 |
| 2.5Y 3.5 0 | 10YR 7 3.5 | 5Y 8.5 1 | 5YR 8 2.5 | 5YR 7.5 3 | 5R 5 6 |
| 10YR 7.5 3 | 5YR 6.5 3.5 | 2.5YR 5 4 | 2.5Y 8 2 | 10YR 8 2 | 2.5Y 7 2 |
| 2.5R 6 6 | 5R 7 6 | 10YR 8 2.5 | 10R 5 6 | N 6.5 0 | 7.5YR 8 3 |

For baseline chip color, the samples are positioned on the sample holder at the intersection of the light paths in the center of the camera view and the chip images are captured. After all chips are imaged, the images are processed using image analysis software (Optimas manufactured by Media Cybernetics, Inc. of Silver Spring, MD). The average RGB values of the chips are extracted by analyzing the stained region of the enamel chip separate from the plastic mounting block.

After treatment, but prior to capturing the samples' images, the system is set to the baseline configuration and calibrated as previously discussed. After calibration, each chip is imaged a second time using the same procedure as before making sure the sample is in the same physical position as the pre-treatment image including orientation of the chip. The images are processed using the image analysis software to obtain the average RGB values of the central four maxillary incisors. The RGB values of all of the images are then mapped into CIE L*′a*b* color space using the RGB values and the L*a*b* values of the color chips on the color standard. The L*a*b* values of the color chips on the color standard are measured using a Photo Research SpectraScan PR650 from Photo Research Inc., LA using the same lighting conditions described for capturing digital images of the facial dentition. The PR650 is positioned the same distance from the color standards as the camera. Each chip is individually measured for L*a*b* after calibration according to the manufacturer's instructions. The RGB values are then transformed into L*a*b* values using regression equations such as:

L*=25.16+12.02*(R/100)+11.75*(G/100)−2.75*(B/100)+1.95*(G/100)³

a*=−2.65+59.22*(R/100)−50.52*(G/100)+0.20*(B/100)−29.87*(R/100)²+20.73*(G/100)²+8.14*(R/100)³−9.17(G/100)³+3.64*[(B/100)²]*[R/100]

b*=−0.70+37.04*(R/100)+12.65*(G/100)−53.81*(B/100)−18.14*(R/100)²+23.16*(G/100)*(B/100)+4.70*(R/100)³−6.45*(B/100)³

The R² for L*, a*, and b* should be >0.95. Each study should have its own equations.

These equations are generally valid transformations in the area of tooth color (60<L*<95, 0<a*<14, 6<b*<25). The data from each chip's set of images is then used to calculate product whitening performance in terms of changes in L*, a* and b*—a standard method used for assessing whitening benefits. When the compositions herein: Changes in L* is defined as ΔL*=L*ₚₒₛₜ₋ₜᵣₑₐₜₘₑₙₜ−L*ₚᵣₑ₋ₜᵣₑₐₜₘₑₙₜ where a positive change indicates improvement in brightness.

After using the whitening products, color changes in CIE Lab color space can be calculated for each sample based on the equations given.

TABLE 2
ChemPC ΔL Values

| | Chemical | | |
|---|---|---|---|
| Examples | Whitening Agent | ΔL | Std Err |
| Ex. 1 | Malonate | 23.97 | 2.06 |
| Ex. 2 | Methylmalonate | 11.18 | 2.00 |
| Ex. 3 | Tartronate | 21.83 | 1.59 |
| Ex. 4 | Malate | 7.79 | 0.80 |
| Ex. 5 | Dimethylmalonate | 6.92 | 0.80 |
| Ex. 6 | Mesoxalate | 16.17 | 1.99 |
| Ex. 7 | DL Tartarate | 5.36 | 0.49 |
| Ex. 8 | L + Tartarate | 5.34 | 0.10 |
| Ex. 9 | Phthalate | 4.89 | 1.07 |
| Ex. 10 | D-Tartarate | 4.39 | 0.67 |
| Ex. 11 | Succinate | 2.08 | 0.19 |
| Ex. 12 | Fumarate | 0.98 | 0.10 |
| Toothpaste Composition 2 | Pyrophosphate | 6.94 | 1.90 |
| Toothpaste Composition 1, pH 5 | None | 2.06 | 0.96 |

TABLE 2 shows the cleaning efficacy of a variety of oral care compositions as illustrated by the ChemPC value. The ChemPC is calculated by comparing the before and after images of a treated stained-bovine chip and determining the change in the *L value in the CIELAB color space. A higher ChemPC value indicates that more stain was removed.

Toothpaste Composition 2 is a whitening toothpaste including sodium acid pyrophosphate (whitening/anticalculus agent). Toothpaste Composition 2 had a ΔL value of about 7. Ex. 1-7 had ChemPC ΔL values of about 7 and higher. The results indicate a significant stain removal potential directly attributed to the dicarboxylate anion

Desirable compositions include those with a ChemPC ΔL value of at least about 7, 8, 9, and/or 10, as determined by the ChemPC method described herein. Desirable compositions also include those where the ChemPC value of the dicarboxylate-containing composition is at least about 2×, 3×, about 4×, or about 5× greater than the pH-matched, dicarboxylate-placebo of the same composition.

One method to assess the potential of dicarboxylic acids to reduce gum disease and improve health was to assess the infection potential of gingival keratinocytes. In this model, human gingival keratinocytes (Gie-No3-B11, purchased from Creative Bioarray, Shirley, NY) were maintained in a 37° C. incubator with 5% CO₂. Cells were seeded in 100 μL cell suspension to each well (2K cells/well). Cells were treated 24 hours after seeding. Growth medium alone or growth medium with solvent vehicle was used as a negative control. Positive controls are either fluorescent E. coli LPS, or fluorescent S. minnesota LPS (https://corporate.thermofisher.com/us/en/index.html), fluorescence-label P. gingivalis outer membrane vesicles (OMV) with or without medium or solvent vehicle. P. gingivalis OMV was isolated following procedures described by Cecil et al. 2016 (Jessica D. Cecil, Neil M. O'Brien-Simpson, Jason C. Lenzo, James A. Holden, Yu-Yen Chen, William Singleton, Katelyn Gause, Yan Yan, Frank Caruso, and Eric C. Reynolds. Differential Responses of Pattern Recognition Receptors to Outer Membrane Vesicles of Three Periodontal Pathogens. iPLoS One. 2016; 11(4): e0151967). The OMV was stained with Lipophilic Tracer DiO following manufacturer's instruction (https://corporate.thermofisher.com/us/en/index.html). Compounds were dissolved in water and diluted in culture medium (all dilutions are deep yellow, or very acidic). All compounds and fluorescent LPS or OMV were added to cells at the same time. It is worth mentioning that cell culture medium changed immediately following adding the compounds from pink (pH7-7.4) to deep yellow (acidic). Images were taken in four sites in each well at a 3 -hour interval over 48 to 120 hours in Incucyte® Live-Cell Analysis Systems (https://www.sartorius.com/en/products/live-cell-imaging-analysis/live-cell-analysis-instruments#id-789214). Data were processed and analyzed using R-programs.

As shown in Table 3 below, dicarboxylic acids can be effective in preventing bacterial virulence factors (lipopolysaccharides and OMV) from infecting human gingival keratinocytes.

TABLE 3
Dicarboxylic Acid Bacterial Virulence

| | Concen- | | Inhibition | |
|---|---|---|---|---|
| Compound | tration | Unit | % | Bacterial virulence |
| Dimethylmalonic_Acid | 5 | mM | 33.8 | E. coli LPS |
| DL_Maleic_acid | 5 | mM | 36.3 | E. coli LPS |
| Fumaric_acid | 7.5 | mM | 31 | E. coli LPS |
| Maleic_acid | 5 | mM | 63.8 | E. coli LPS |
| Malonic_acid | 5 | mM | 35.9 | E. coli LPS |
| Methyl_malonic_acid | 5 | mM | 30.7 | E. coli LPS |
| Oxalic_acid | 125 | μM | 38.2 | E. coli LPS |
| Potassium_oxalate_monohydrate | 125 | μM | 21.5 | E. coli LPS |
| Dimethylmalonic_Acid | 11.11 | mM | 81.3 | P. gingivalis OMV |
| DL_Maleic_acid | 11.11 | mM | 62.2 | P. gingivalis OMV |
| DL_Tartaric_acid | 1 | mM | 57.7 | P. gingivalis OMV |
| Fumaric_acid | 33.33 | mM | 53.1 | P. gingivalis OMV |
| L_+_Tartaric_acid | 2.5 | mM | 73.6 | P. gingivalis OMV |
| Maleic_acid | 100 | mM | 83.3 | P. gingivalis OMV |
| Malonic_acid | 11.11 | mM | 69.3 | P. gingivalis OMV |
| Methyl_malonic_acid | 11.11 | mM | 63.6 | P. gingivalis OMV |
| Oxalic_acid | 100 | mM | 32.8 | P. gingivalis OMV |
| Sodium_mesoxalate_monohydrate | 5 | mM | 44.3 | P. gingivalis OMV |
| Succinic_acid | 2 | mM | 73 | P. gingivalis OMV |
| Dimethylmalonic_Acid | 7.5 | mM | 54.5 | S. minnesota LPS |
| DL_Maleic_acid | 7.5 | mM | 59.9 | S. minnesota LPS |
| DL_Tartaric_acid | 1 | mM | 26.4 | S. minnesota LPS |
| Fumaric_acid | 7.5 | mM | 71.9 | S. minnesota LPS |
| L_+_Tartaric_acid | 10 | mM | 52.1 | S. minnesota LPS |
| Maleic_acid | 3 | mM | 73.8 | S. minnesota LPS |
| Methyl_malonic_acid | 2.5 | mM | 28.7 | S. minnesota LPS |
| Oxalic_acid | 125 | μM | 22.5 | S. minnesota LPS |
| Potassium_oxalate_monohydrate | 125 | μM | 41.5 | S. minnesota LPS |
| Succinic acid | 1 | mM | 42.3 | S. minnesota LPS |

### Example 2

### Preparation of Oral Care Composition

The oral care composition of TABLE 4 was prepared by combining one or more humectants, water, sweetener(s), and whitening agent(s) to create a liquid mixture. The liquid mixture was homogenized at 25° C. until homogeneous and completely dissolved. Next, sodium hydroxide (50% solution) was added to the liquid mixture and the liquid mixture was homogenized at 25° C. until homogeneous and completely dissolved. A separate powder mixture was prepared by combining the abrasive silica, thickening silica, and opacifier, with any thickening agents, such as xanthan gum and/or sodium carboxymethylcellulose. The powder mixture was then combined with the liquid mixture and homogenized completely. Next, the surfactant, such as sodium lauryl sulfate, and flavor was added to the mixture. The contents were homogenized at 25° C. until homogeneous and entrained air was removed by vacuum.

TABLE 4
Oral Care Composition

| Ingredient (wt %) | Ex. 14 |
|---|---|
| Sorbitol | 64.000 |
| Treated Water | 6.595 |
| NaOH (50% Solution) | 1.525 |
| Saccharin | 0.400 |
| Xanthan Gum | 0.500 |
| Carboxymethylcellulose | 1.000 |
| Malonic Acid | 1.775 |
| TiO₂ | 0.500 |
| Silica Thickening Type | 0.500 |
| Silica Dental Abrasive Type | 17.000 |
| Sodium Lauryl Sulfate | 5.000 |
| (28 wt % solution) | |
| Flavor | 1.205 |
| Total | 100.000 |

### Clinical Whitening and Oral Irritation Evaluation

A randomized controlled trial was used to evaluate naturally occurring extrinsic tooth stain removal over a one-week period. After institutional review and informed consent, adult volunteers with natural extrinsic tooth stain were randomly assigned to either Ex. 14, to Crest 3D White Brilliance toothpaste positive control, or to Colgate Cavity Protection negative control all using their own toothbrush. Table 5 shows the ingredients of Crest 3D White Brilliance and Colgate Cavity Protection. Stain was assessed on the facial surfaces of anterior teeth using the Interproximal Modified Lobene Stain Index (IMLSI) after one week of product use. Safety was assessed via clinical examination and interview. Analysis of covariance was used to compare between the three groups. The statistical comparisons were two-sided using a 5% level of significance.

TABLE 5
Oral Care Compositions

| Positive Control | Negative Control |
|---|---|
| Crest 3D White Brilliance | Colgate Cavity Protection |
| Glycerin | Dicalcium Phosphate |
| | Dihydrate |
| Dental Silica | Water |
| Sodium Polyphosphate | Glycerin |
| Water | Sodium Lauryl Sulfate |
| PEG-6 | Cellulose Gum |
| Flavor | Flavor |
| Trisodium Phosphate | Tetrasodium Pyrophosphate |
| Sodium Lauryl Sulfate | Sodium Saccharin |
| Carrageenan | Sodium |
| | Monofluorophosphate |
| Cocamidopropyl Betaine | |
| Sodium Saccharin | |
| PEG-20M | |
| Xanthan Gum | |
| Sucralose | |
| Mica | |
| Titanium Dioxide | |
| Sodium Fluoride | |

To qualify for the study, subjects had to have a mean score of greater than or equal to 1.5 for the test teeth, be at least 18 years of age, be in good general health, and agree not to participate in any non-study dentistry. Subjects with severe periodontal disease, fixed orthodontics, or other diseases or conditions deemed by the investigator to potentially interfere with the subject's participation in the study were excluded. Qualifying subjects were then stratified based on the composite stain scores of the test teeth, gender, and age. Within strata, subjects were randomly assigned to one of three treatment groups using an encoded program.

Safety was assessed at Baseline and Week 1 by examining the oral soft tissues (OST). The OST exam consisted of a visual intra-oral and peri-oral examination using standard dental unit lighting, a dental mirror, and gauze. The intra-oral examination included an assessment of all intra-oral soft tissue structures.

The area (extent) and intensity of dental stain was scored on the facial surfaces of the test teeth, with each of these surfaces divided into gingival, interproximal, and body regions, and scored separately. The gingival region for the IML Stain Index is defined as an approximately 2 mm-wide band on the labial surface of the enamel adjacent to the cemento -enamel junction, and the interproximal (mesial and distal) regions as bands approximately 2 mm in width bounded by the gingival region and the contact point approximal to the proximal line angle. The body is defined as the remainder of the surface. The extent of stain was scored as follows: 0=no stain detected; 1=stain covering up to ⅓ of the region; 2=stain covering>⅓ and up to ⅔ of the region; and 3=stain covering>⅔ of the region. The intensity of stain was scored as follows: 0=no stain; 1=light stain (yellow to light brown or gray); 2=moderate stain (medium brown); and 3=heavy stain (dark brown to black). A score of 8 was used for non-gradable sites, and a score of 9 for a missing anterior tooth. These sites were not included in the statistical analyses. The IML Stain Index area and intensity scores were calculated for each subject by averaging the area or intensity score for a region (sum of all respective scores/all sites graded). The composite score was calculated by averaging the sum of all scores (sum of area times intensity scores/all sites graded), with the interproximal score calculated using only mesial and distal regions.

Statistical analyses for the primary efficacy outcome were based on the IML Stain Index changes from Baseline minus Week 1 measurements and were analyzed using two-sided paired t-tests and a significance level of 0.05. Treatment differences, the secondary outcome, were assessed using an analysis of covariance with baseline stain score as the covariate. In addition, the percent IML stain reduction was calculated for each subject as 100%×(baseline stain score minus post-baseline stain score)/baseline stain score. The median of these percent stain reductions was calculated for each group.

TABLE 6
Interproximal Modified Lobene Composite Stain Index on All Evaluable Surfaces

| | | | | 2-sided p-value | |
|---|---|---|---|---|---|
| | | Adjusted Wk 1 | % Diff vs. | Positive | |
| Treatment | N | Mean (SE*) | Neg Ctrl | Control | Ex. 14 |
| Negative Control | 29 | 1.38 (0.057) | — | < 0.001 | < 0.001 |
| Positive Control | 30 | 1.00 (0.056) | − 27.76 | | 0.195 |
| Ex. 14 | 30 | 1.10 (0.056) | −20.28 | | |

*SE = standard error of measurement

The IML composite tooth stain scores, percent difference vs. negative control, and the 2 -side p-value comparisons are given TABLE 6. The positive control (Crest 3D White Brilliance) provided statistically significant reductions in naturally occurring extrinsic tooth stain with respect to the negative control (Colgate Cavity Protection). Ex. 14 also provided statistically significant whitening with respect to the negative control while not being statistically significantly different than the positive control. Both whitening toothpastes, therefore, achieved a statistically significant reduction in naturally occurring intrinsic tooth stain over the one-week period of the study.

TABLE 7
Adverse Events Possibly Related to Product Use of All Subjects

| | | | | Reported/ |
|---|---|---|---|---|
| Treatment | General Term | Specific Term | Severity | Observed |
| Positive Control | Oral Mucosal Exfoliation | Desquamation Mouth | Mild | Both |
| Positive Control | Oral Mucosal Exfoliation | Desquamation Mouth | Mild | Reported |
| Positive Control | Oral Mucosal Exfoliation | Desquamation Mouth | Mild | Reported |
| Positive Control | Gingival Discomfort | Irritation Gum | Mild | Reported |
| Positive Control | Oral Mucosal Exfoliation | Desquamation Mouth | Mild | Reported |
| Positive Control | Oral Mucosal Exfoliation | Desquamation Mouth | Mild | Reported |
| Positive Control | Oral Mucosal Exfoliation | Desquamation Mouth | Mild | Reported |
| Ex. 14 | Hyperaesthesia Teeth | Sensitivity of Teeth | Mild | Reported |

The results of the OST assessment following product use are given in TABLE 7. The positive control (Crest 3D White Brilliance) produced more adverse events than Ex. 14. The positive control produced nine mild adverse events that were reported by the subject, observed by the examiner, or both. Eight of the nine adverse events for Crest 3D White Brilliance were reported to be oral mucosal exfoliation by mouth desquamation, while one event was reported as gingival discomfort as an irritated gum. By contrast, Ex. 14 produced only a single mild adverse event, hyperaesthesia teeth/tooth sensitivity. For the positive control, 9 out of 29 subjects or approximately 31% of subjects experienced an adverse event probably related to product use. For Ex. 14, 1 out of 30 subjects or approximately 3% of subjects experienced an adverse event probably related to product use. No adverse events were reported for the negative control (Colgate Cavity Protection). Unexpectedly, users of Ex. 14 reported significantly fewer instances of adverse event probably related to the tooth product while they experience not statistically significantly different whitening with respect to the positive control.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A whitening dentifrice composition comprising:
dicarboxylic acid, the dicarboxylic acid comprising malonic acid, methylmalonic acid, dimethylmalonic acid, dihydroxymalonic acid, salts thereof, or combinations thereof,
wherein the whitening dentifrice composition has a ChemPC ΔL of at least about 7, and
wherein the whitening dentifrice composition is essentially free of, substantially free of, or free of polyphosphate.

2. The composition of claim 1, wherein the ChemPC ΔL is at least about 10.

3. The composition of claim 1, wherein the dicarboxylic acid comprises malonic acid or a salt thereof.

4. The composition of claim 1, wherein the whitening dentifrice composition is free of polyphosphate, peroxide, chlorohexidine, and bicarbonate.

5. The composition of claim 1, wherein the pH is from about 4 to 5.

6. The composition of claim 1, wherein the whitening dentifrice comprises greater than 2% to about 10%, by weight of the composition, of the dicarboxylic acid.

7. The composition of claim 1, further comprising metal, wherein the metal comprises tin, zinc, copper, potassium, or combinations thereof.

8. The composition of claim 7, wherein the tin comprises stannous fluoride, stannous chloride, or combinations thereof.

9. The composition of claim 7, wherein the zinc comprises zinc citrate, zinc lactate, zinc oxide, zinc phosphate, or combinations thereof.

10. The composition of claim 7, wherein the potassium comprises potassium nitrate.

11. The composition of claim 1, wherein the composition is free of potassium.

12. The composition of claim 1, further comprising fluoride, wherein the fluoride comprises stannous fluoride, sodium fluoride, sodium monofluorophosphate, amine fluoride, or combinations thereof.

13. The composition of claim 1, further comprising up to about 45%, by weight of the composition, of water.

14. The composition of claim 1, further comprising thickening agent, wherein the thickening agent comprises polysaccharide, polymer, silica thickener, or combinations thereof.

15. The composition of claim 1, further comprising abrasive, wherein the abrasive comprises silica abrasive, calcium abrasive, or combinations thereof.

16. The composition of claim 15, wherein the composition comprises less than 5%, by weight of the composition, of the abrasive.

17. The composition of claim 15, wherein the composition comprises from about 0.5% to about 2%, by weight of the composition, of the abrasive.

18. The composition of claim 1, wherein the composition is essentially free of, substantially free of, or free of abrasive.

19. A mouth rinse composition comprising:
(a) dicarboxylic acid, the dicarboxylic acid comprising malonic acid, methylmalonic acid, dimethylmalonic acid, dihydroxymalonic acid, salts thereof, or combinations thereof; and
(b) at least about 75%, by weight of the composition, of water,
wherein the composition has a ChemPC ΔL of least about 7, and
wherein the whitening dentifrice composition is essentially free of, substantially free or free of polyphosphate.

20. An oral care composition comprising:
(a) dicarboxylic acid, the dicarboxylic acid comprising malonic acid, methylmalonic acid, dimethylmalonic acid, dihydroxymalonic acid, salts thereof, or combinations thereof; and
(b) fluoride,
wherein the whitening dentifrice composition is essentially free of, substantially free of, or free of polyphosphate.
